(19) United States
(12) Patent Application Publication
(10) Pub. No.: US 2003/0078633 A1
(43) Pub. Date: Apr. 24, 2003

(54) Methods and implantable apparatus for electrical therapy

(76) Inventors: Andrew D. Firlik, Ridgefield, CT (US); Alan J. Levy, Bellevue, WA (US); Bradford Evan Gliner, Sammamish, WA (US)

Correspondence Address: PERKINS COIE LLP, PATENT-SEA, P.O. BOX 1247, SEATTLE, WA, 98111-1247, US

(21) Appl. No.: 10/260,699

(22) Filed: Sep. 30, 2002

Related U.S. Application Data
(60) Provisional application No. 60/325,982, filed on Sep. 28, 2001.

(51) Int. Cl. A61N 1/18

(52) U.S. Cl. 607/46

(57) ABSTRACT

Some embodiments of the invention provide electrical therapy by delivering electrical pulses through at least one (and in some embodiments two or more) subcutaneously implanted electrode to stimulate one or more target nerves in a selected volume of tissue. Unlike conventional peripheral nerve stimulation or spinal column stimulation, there is no need to position the electrodes in direct contact with a specific nerve or the spinal column. Instead, the electrode can be implanted at an indeterminate distance from the target nerve. Other embodiments provide implantable neurostimulators with a pair of electrodes which can be connected to a common pulse system. Each electrodes may include two or more contacts spaced 3 cm or more from one another and the electrodes are implantable at distances of 5 cm or more apart. The common pulse system may also be implantable.

### CROSS-REFERENCE TO RELATED APPLICATION(S)

This application claims the benefit of U.S. Application No. 60/325,982 filed Sep. 28, 2001.

### TECHNICAL FIELD

The present invention generally relates to methods and devices for electrical therapy, such as neurostimulation. In particular, the present invention provides devices and methods for subcutaneous electrical therapy.

### BACKGROUND

Electrical therapy has been used for a number of years to treat pain and other conditions. For example, transcutaneous electrical nerve stimulation (“TENS”) systems deliver electrical energy via electrode patches placed on the surface of a patient's skin. Electrical power is delivered to the patches to treat pain in tissue beneath and around the location of the patches. One problem with TENS systems is that electrical stimulation must pass through the patient's skin before reaching nerves located deeper within the patient's body. To deliver adequate stimulation to the deeper nerves, the intensity of the stimulation in nerves located in the skin near the patches may reach painful levels. As a consequence, TENS systems may not provide patients with adequate pain relief.

Percutaneous neuromodulation therapy (“PNT,” also sometimes called percutaneous electrical nerve stimulation or “PENS”) employs percutaneously placed electrodes to deliver electrical stimulation. PNT has proven to deliver significantly better pain relief than TENS treatments using skin surface electrodes. PNT is described in a number of articles, including: Ghoname, et al., “Percutaneous Electrical Nerve Stimulation for Low Back Pain,” JAMA 281: 818-823 (1999); Ghoname, et al., “The Effect of Stimulus Frequency on the Analgesic Response to Percutaneous Electrical Nerve Stimulation in Patients with Chronic Low Back Pain,” Anesth. Analg. 88: 841-6 (1999); White, et al., “Percutaneous Neuromodulation Therapy: Does the Location of Electrical Stimulation Effect the Acute Analgesic Response?,” Anesth. Analg. 91: 1-6 (2000); and White, et al., “The Effect of Montage on the Analgesic Response to Percutaneous Neuromodulation Therapy,” Anesth. Analg. 92: 483-7 (2001). These articles are incorporated herein by reference. Techniques and devices for positioning electrodes for PNT are disclosed in PCT International Publication No. WO 01/39829 by Vertis Neuroscience, Inc., which is also incorporated herein by reference.

One disadvantage of PNT is the array of relatively rigid electrodes extending through the patient's skin. Each of these electrodes is separately connected to a power supply by a separate cable. This effectively precludes a patient from moving freely during PNT sessions. If the PNT session is kept relatively short, e.g., on the order of an hour or less, this may be a relatively minor inconvenience. However, longer-term treatment can be more problematic. At the end of a session, the electrodes must be removed to allow a patient to go about normal daily activities without undo hindrance. If the patient is to undergo multiple therapy sessions, the electrodes would need to be repositioned for each new session. Absent tattooing or the like, it can be difficult to ensure consistent percutaneous placement of the electrodes. PNT also requires an external power source, which is typically retained by the treating physician, requiring the patent to visit the physician's offices or a clinic for each treatment session.

Another technique for electrically treating patients involves the permanent placement of a neurostimulator in the patient's body. Such implantable neurostimulators are commercially available, e.g., from Medtronic, Inc. of Minneapolis, Minn., U.S. These neurostimulators have been used only in two highly specific applications—spinal cord stimulation (“SCS”) and peripheral nerve stimulation (“PNS”). In SCS, an electrical lead is positioned within the epidural space in direct electrical contact with the dura mater. The electrical leads may be positioned in the epidural space percutaneously or by direct surgical intervention wherein the physician positions a paddle with an array of electrodes directly against the dura mater. By positioning the lead at a particular location along the patient's spine, an analgesic or paresthetic effect can be achieved in a relatively large portion of the patient's body associated with the selected location. For example, positioning the lead dorsally on the spinal column at any one of vertebral levels T1-T3 can deliver broad paresthesia to the patient's arms. In addition to the relatively broad, unfocused target areas associated with SCS, accessing the patient's epidural space to position the leads risks dangerous infections or damage to the spine itself.

PNS is similar to SCS, but a lead is placed in direct electrical contact with a peripheral nerve rather than contacting the spinal column. This requires a physician to surgically expose the nerve to be treated, such as the sciatic nerve, and physically abut the lead against that nerve surface. Some PNS leads are designed as electrical collars that must be wrapped around the nerve. By appropriate selection of the peripheral nerve to be treated, PNS can be used to deliver more localized effects than SCS. However, PNS requires relatively extensive and invasive surgery to identify the specific location of the nerve to be treated and the electrode must be carefully positioned in direct contact with the exposed nerve.

### BRIEF DESCRIPTION OF THE DRAWINGS

FIG. 1 schematically illustrates subcutaneous implantation of a pair of electrodes for treating a wound in accordance with one embodiment of the present invention.

FIG. 2 schematically illustrates subcutaneous implantation of a pair of electrodes and a pulse system for electrical therapy in accordance with another embodiment of the invention.

FIG. 3 schematically illustrates subcutaneous implantation of four electrodes and a pulse system for electrical therapy in accordance with another embodiment of the invention.

FIG. 4 schematically illustrates subcutaneous implantation of a single electrode and a pulse system for electrical therapy in accordance with another embodiment of the invention.

FIG. 5 is a cross-sectional view illustrating an electrode in accordance with one embodiment of the invention.

FIG. 6A is a cross-sectional view of an electrode in accordance with an alternative embodiment of the invention.

FIG. 6B is a cross-sectional view of the electrode of FIG. 6A taken along line B-B.

FIG. 7 is an isometric elevation view of an electrode in accordance with another embodiment of the invention.

FIG. 8 is a cross-sectional view of an electrode in accordance with yet another embodiment of the invention.

FIG. 9 is a cross-sectional view of an electrode in accordance with still another embodiment of the invention.

FIG. 10 is a schematic illustration of an implantable stimulation apparatus having a pulse system and an external controller in accordance with one embodiment of the invention.

FIG. 11 is a schematic illustration of an implantable stimulation apparatus having a pulse system and an external controller in accordance with another embodiment of the invention.

### DETAILED DESCRIPTION

Various embodiments of the present invention provide implantable electrical stimulators and methods for delivering electrical therapy using implantable electrical stimulators. The following description provides specific details of certain embodiments of the invention illustrated in the drawings to provide a thorough understanding of those embodiments. It should be recognized, however, that the present invention can be reflected in additional embodiments and the invention may be practiced without some of the details in the following description.

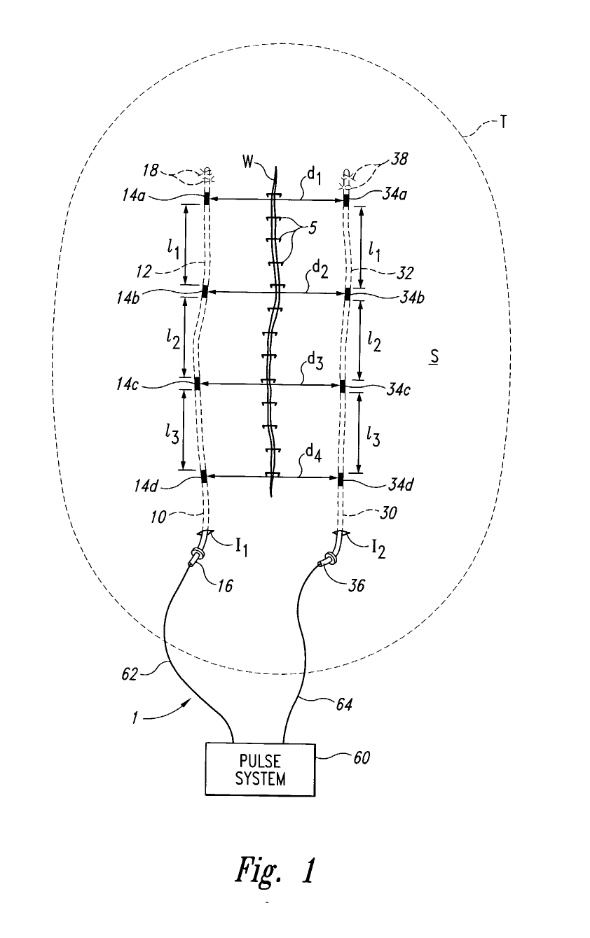

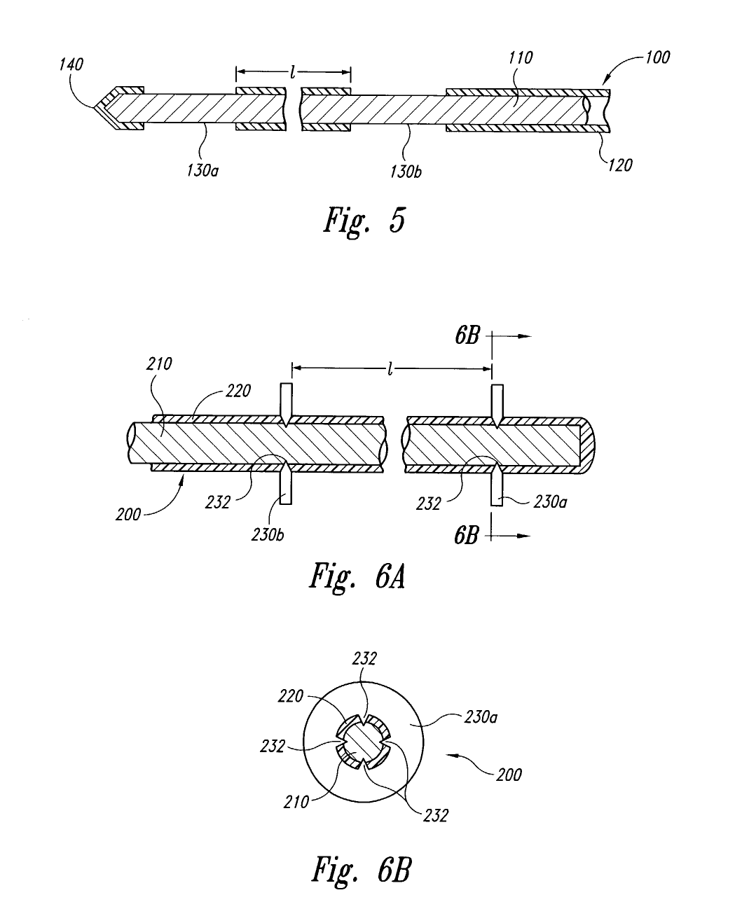

The operation and features of neurostimulators and neurostimulator components in accordance with certain aspects of the invention are best understood in light of exemplary uses for these devices. Several methods of electrode treatment in accordance with embodiments of the invention are, therefore, discussed first in connection with FIGS. 1-4. The details and features of selected embodiments of neurostimulator components are discussed with reference to FIGS. 5-11.

A. Methods of Delivering Electrical Therapy in Accordance with Selected Embodiments of the Invention

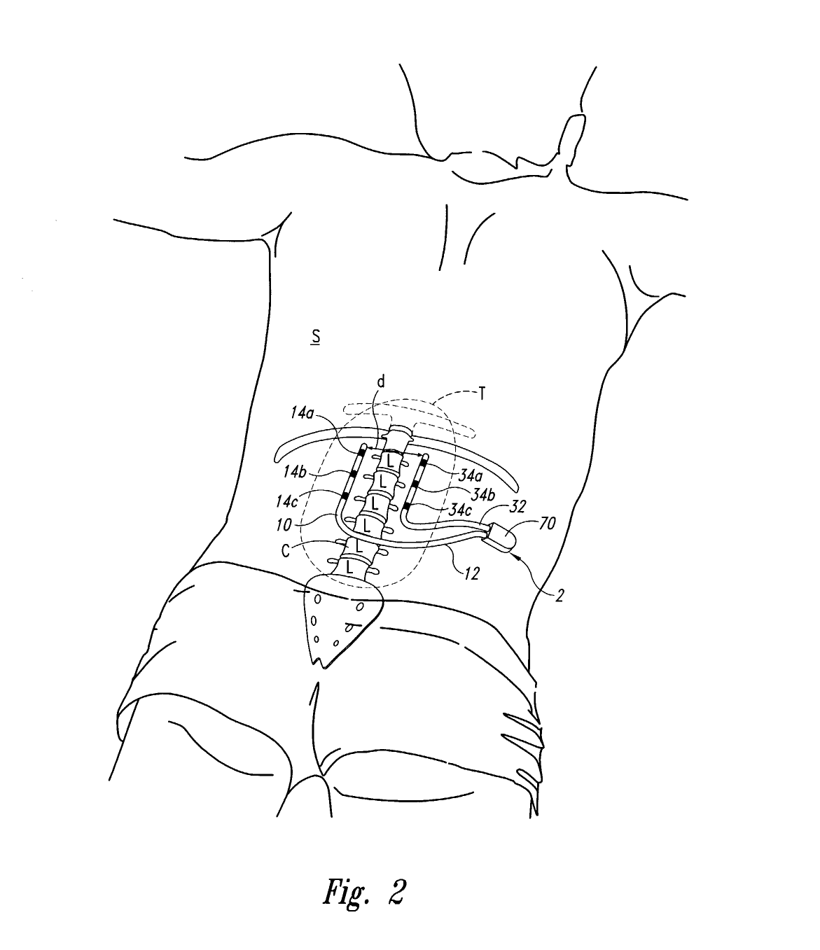

FIG. 1 schematically illustrates a pair of electrodes 10, 30 implanted for electrically therapy of a wound W in a tissue volume T. In FIG. 1, the wound W is typified as a surgical incision closed with a series of staples 5, but the wound W need not be a surgical incision. As noted below in connection with FIG. 2, electrically therapy in accordance with other aspects of the invention involve implanting electrodes on opposite sides of a patient's spinal column. If so desired, these techniques may be combined, e.g., where the wound W is a surgical incision made in the course of a surgical procedure on the back, such as fusing vertebrae or treating vertebral discs.

The first electrode 10 will be subcutaneously implanted along a first side of the wound W and the second electrode 30 will be subcutaneously implanted along a second side of the wound W. The first electrode 10 includes a flexible elongate body 12 carrying at least one electrical contact 14. In one desirable embodiment, the electrode 10 includes two or more contacts 14 spaced along the length of the body 12. In particular, a first contact 14a may be located adjacent a distal end of the electrode 10 with a second contact 14b spaced a distance l₁ from the first contact 14a. A third contact 14c is spaced a distance l₂ along the length of the body 12 from the second contact 14b. A fourth contact 14d is spaced a distance l₃ along the length of the body 12 from the third contact 14c. While the first electrode 10 is shown as including four contacts 14, it should be understood that more or fewer contacts could be employed, depending on the length of the wound W and the electrical power to be delivered to each of the contacts 14.

The lengths l₁-l₃ of the body 12 between the contacts 14 can be varied to achieve a desired objective. In one embodiment of the invention, each of the lengths l₁-l₃ is at least 3 cm, e.g., 3-15 cm. In one further embodiment, the lengths l₁-l₃ are all 4-10 cm; a length l₁-l₃ between adjacent contacts of 4-5 cm is believed appropriate for a number of applications. For shorter wounds W, these lengths could be less than 3 cm and/or fewer than four contacts 14 could be employed. While the lengths l₁-l₃ in FIG. 1 are shown as being approximately equal to one another, the length of the body 12 between adjacent pairs of contacts may be varied.

The first electrode 10 may be implanted beneath the skin S to extend along the first side of the wound W in any desired fashion. For example, the first electrode 10 can be surgically implanted at the desired location within the tissue volume T by making a lateral incision from the site of the wound W and placing the electrode 10 in place before closing the wound W with the staples 5. In an alternative embodiment, the first electrode 10 is delivered to the desired site via a percutaneous procedure. In such a procedure, a small incision I₁ can be made in the skin spaced laterally away from the wound W. The first electrode 10 can then be introduced beneath the skin S through the incision I₁ and advanced distally to lie lengthwise along the side of the wound W. In one embodiment, the electrode 10 may have sufficient column strength or “pushability” to allow it to be advanced through the tissue volume T to the desired location.

In other embodiments of the invention wherein the first electrode 10 lacks the necessary pushability, other techniques may be employed. In one such technique, a stylet is introduced into a hollow lumen of the electrode 10 to provide the necessary column strength and the stylet and electrode 10 are advanced to the desired location. Thereafter, the stylet may be removed, leaving the electrode 10 in place. Another embodiment of the invention employs a trocar having a hollow lumen sized to receive the electrode 10. The trocar may comprise a hollow stainless steel tube, for example. The trocar and electrode 10 may be advanced through the tissue volume T to the desired location. The trocar can then be withdrawn, leaving the electrode 10 in place. The tissue volume T will tend to close back around the electrode 10, ensuring good physical contact of the tissue against the contacts 14.

In the schematic illustration of FIG. 1, the first electrode 10 is illustrated as extending through the patient's skin S through the incision I₁. An electrical connector 16 is provided at the exposed proximal end of the electrode 10. This connector 16 may comprise any plug or receptacle suitable for connection to a pulse system 60, discussed below.

As shown in FIG. 1, the first electrode 10 may be held in place by an anchor 18. The anchor 18 in FIG. 1 is typified as a pair of sutures, which may be passed through the skin S and through a distal length of the electrode 10. Such an anchor 18 can limit undesirable movement of the electrode 10 and reduce the chances that the electrode 10 will be prematurely dislodged from the tissue volume T. To facilitate removal of the electrode 10 upon completion of a treatment regime, the anchor 18 may be a temporary anchor. If the anchor 18 comprises sutures, as shown, the sutures may be absorbable sutures selected to have a suitable absorption time. The temporary anchor 18 could, instead, be a selectively controllable anchor. For example, the anchor 18 may comprise a balloon carried adjacent the distal end of the electrode 10 which may be inflated via an inflation lumen to anchor the electrode in place and then subsequently deflated through the same inflation lumen when the treatment regime is completed. Alternatively, the temporary anchor may be a selectively deployable barb, which may take the form of a curved wire that is deployed laterally through the wall of the electrode 10, as discussed more fully below in connection with FIG. 9.

In certain embodiments of the invention, only a single electrode 10 may be employed, without need for a second electrode 30. The embodiment of FIG. 1, however, employs a second electrode 30, which may have a structure similar to that of the first electrode 10. For example, the second electrode 30 may have a body 32 with a first contact 34a adjacent its distal end, a second contact 34b spaced a length l₁ along the body 32 from the first contact 34a, a third contact 34c spaced a length l₂ along the body 32 from the second contact 34b, and a fourth contact 34d spaced a third length l₃ along the body 32 from the third contact 34c. A connector 36 may be provided adjacent the proximal end of the second electrode 30 to facilitate attachment to the pulse system 60. The second electrode 30 may be implanted in the tissue volume T in much the same manner as discussed above for the first electrode 10. The second electrode 30 may be held in place after implantation by an anchor 38.

As noted above, the first electrode 10 is positioned on a first side of a wound W and the second electrode 30 is positioned on a second side of a wound W. In the illustrated embodiment, the first and second electrodes have a similar structure and the same number of contacts along their respective lengths. The contacts 34 of the second electrode 30 may be positioned laterally directly across the wound W from the contacts 14 of the first electrode 10, as shown. In another embodiment (not shown), the contacts 34 of the second electrode 30 are instead staggered with respect to the contacts 14.

The distances between the first and second electrodes 10, 30 can be varied to affect the density of the electrical field adjacent the wound W and within the balance of the tissue volume T. In the arrangement shown in FIG. 1, the first contact 14a of the first electrode 10 is spaced a distance d₁ from the first contact 34a of the second electrode 30. Similarly, the second contacts 14b and 34b are separated by a distance d₂, the third contacts 14c and 34c are separated by a distance d₃, and the fourth contacts 14d and 34d are separated by a distance d₄. The distances d₁-d₄ may be approximately the same, as shown. Alternatively, distances d₁-d₄ may be varied so that some of the adjacent pairs of contacts (e.g., 14b and 30b) are spaced closer together than are other adjacent pairs of contacts (e.g., 14a and 34a). In one embodiment, each of the contacts is spaced at least 2.5 cm from the wound W, with a range of 2.5-10 cm being appropriate for many applications. In this embodiment, the distances d₁-d₄ may be about five to about 20 cm, e.g., about 6-12 cm.

The electrodes 10, 30 can be positioned in any desired subcutaneous tissue. In one embodiment of the invention, both of the electrodes 10, 30 are positioned in subcutaneous fat, lying between the skin S and underlying fascia and muscle. As compared to the conductive pads applied on top of the skin in common TENS systems, placing the electrodes 10 and 30 in the subcutaneous fat positions the contacts 14 and 34 closer to subcutaneous nerves and avoids the necessity of transmitting power through the nerve-dense skin S to treat these subcutaneous nerves. As a consequence, performance of the electrodes 10 and 30 positioned in subcutaneous fat is anticipated to yield more effective pain reduction than is commonly achievable with TENS systems.

In another embodiment of the invention, each of the electrodes 10 and 30 are positioned in muscle tissue instead of subcutaneous fat. This will position the contacts 14 and 34 deeper in the tissue volume T, enhancing stimulation of subcutaneous nerves while better avoiding over stimulation of nerves in the skin S.

The first electrode 10 and the second electrode 30 may be electrically coupled to a common pulse system 60. In the embodiment of FIG. 1, the pulse system 60 is positioned extracorporeally. The connector 16 of the first electrode may be coupled to the pulse system 60 by a first extension 62 and the connector 36 of the second electrode 30 may be coupled to the pulse system 60 by a second extension 64.

The pulse system 60 is adapted to deliver a controlled series of electrical pulses at power levels and a waveform efficacious to reduce the sensation of pain in subcutaneous nerves contained within the tissue volume T. In one embodiment, the pulse system 60 supplies a current-regulated and current-balanced waveform with an amplitude of up to 15 milliamperes, a frequency of approximately 4 Hz to approximately 100 Hz, and a pulse width of approximately 50 microseconds to approximately 1 millisecond. In adaptation of this embodiment, the amplitude of the electrical power is about 0.6-8 milliamperes, with a range of 0.7-7.8 milliamperes being useful for sustained treatment in a wide variety of patients. A power level of about 2-3 milliamperes, e.g., about 2.5 milliamperes, is expected to be efficacious for many patients. One pulse system 60 expected to be useful in connection with the present invention is disclosed in U.S. patent application Ser. No. 09/751,503, entitled “System and Method for Varying Characteristics of Electrical Therapy,” filed Dec. 29, 2000 and incorporated herein by reference.

The neurostimulator 1 of FIG. 1 (i.e., the first electrode 10, the second electrode 30, and the pulse system 60) can be used to manage pain associated with the wound W. The wound W will cause pain in a number of cutaneous and subcutaneous nerves positioned within the tissue volume T. Subcutaneously implanting the electrodes 10 and 30 adjacent to the wound W permits the subcutaneous nerves and nerve ends adjacent the wound W to be electrically treated to reduce the sensation of pain in those nerves. This encompasses not only the nerve ends and severed nerves exposed in the wound W, but also the nerve branches containing those nerve ends and severed nerves, as well as the surrounding nerve endings. This provides a relatively broad paresthesia in the tissue volume T going beyond the mere surface of the wound W. Even so, the area of paresthesia can remain relatively focused on the site of the wound W.

This ability to stimulate a selected tissue volume T adjacent the site of the wound W provides benefits not readily realized by conventional spinal cord stimulation (SCS) or peripheral nerve stimulation (PNS). If SCS were employed to reduce pain associated with a wound W, this would require invasion of the epidural space with its attendant risks. The paresthesia achieved with SCS will also be relatively broad, involving tissue significantly beyond the nerves involved in the wound W. The use of PCS to manage pain associated with the wound W is even more problematic. Typically, the wound W will involve a number of nerve endings and nerve branches rather than a single main nerve. As a consequence, a physician would have to perform a separate procedure to track the nerves involved in the wound W back to a common root and contact an electrode to the root nerve. Not only is this unnecessarily invasive, it likely will also electrically stimulate nerves associated with the root nerve which go well beyond the specific nerves involved in the wound W.

This embodiment of the invention can be particularly useful in connection with managing post-operative pain associated with a surgical procedure. In one such application of this embodiment, the electrodes 10 and 30 can be subcutaneously implanted adjacent the incision W while the patient is still under general or local anesthesia used for the surgical procedure. As the anesthesia wears off, the neurostimulator 1 can be used to reduce the sensation of pain associated with the wound.

The electrodes 10 and 30 may be removed from the tissue volume T at the end of a treatment period. The treatment period may comprise a relatively short post-operative period (e.g., 6 hours or less) to manage the pain of the wound W after the anesthesia wears off and before the level of pain is readily managed by oral analgesics or the like. In another embodiment of the invention, the electrodes 10 and 30 can remain in place for a much longer period of time. In one embodiment useful for patients undergoing back surgery, the electrodes 10 and 30 may remain in place for three weeks or longer. The connectors 16 and 36 of the electrodes may be coupled to the pulse system 60 for a series of separate treatment sessions ranging from about 30 minutes to about four hours. Between treatment sessions, the connectors 16 and 36 may be disconnected from the pulse system 60, enabling the patient to move about more freely. If the connectors 16 and 36 remain extending through the patient's skin S, as shown, these connectors 16 and 36 may be taped or otherwise held in place between treatment sessions.

The benefits of the present invention are not limited to electrical therapy of wounds W. Another embodiment of the invention provides a long-term treatment for pain in various areas of the body. FIG. 2, for example, illustrates one particular application of an embodiment of the invention for use in treating low back pain, e.g., pain associated with degenerative discs. Many of the elements of the neurostimulator 1 of FIG. 1 can be used in the neurostimulator 2 of FIG. 2. Hence, the neurostimulator 2 shown in FIG. 2 includes a first electrode 10 and a second electrode 30. The first electrode 10 has a body 12 with a plurality of contacts 14a-c spaced along a predetermined length of the electrode 10. Similarly, the second electrode 30 has a body 32 with a plurality of contacts 34a-c spaced along a predetermined length of the second electrode 30. The electrodes 10 and 30 of FIG. 2 employ only three contacts each rather than the four contacts shown in FIG. 1, but the electrodes 10 and 30 in the two drawings may otherwise be substantially the same.

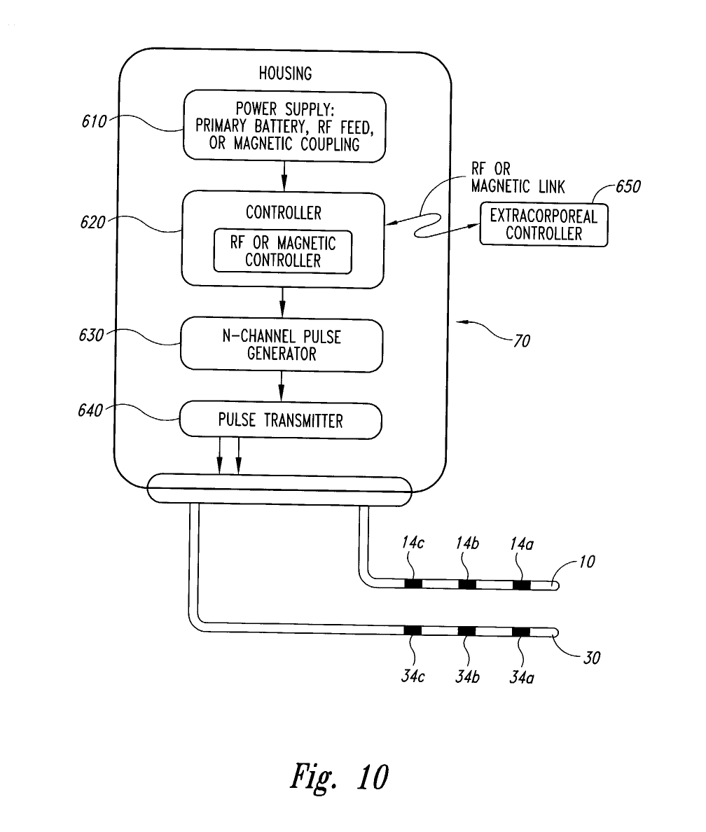

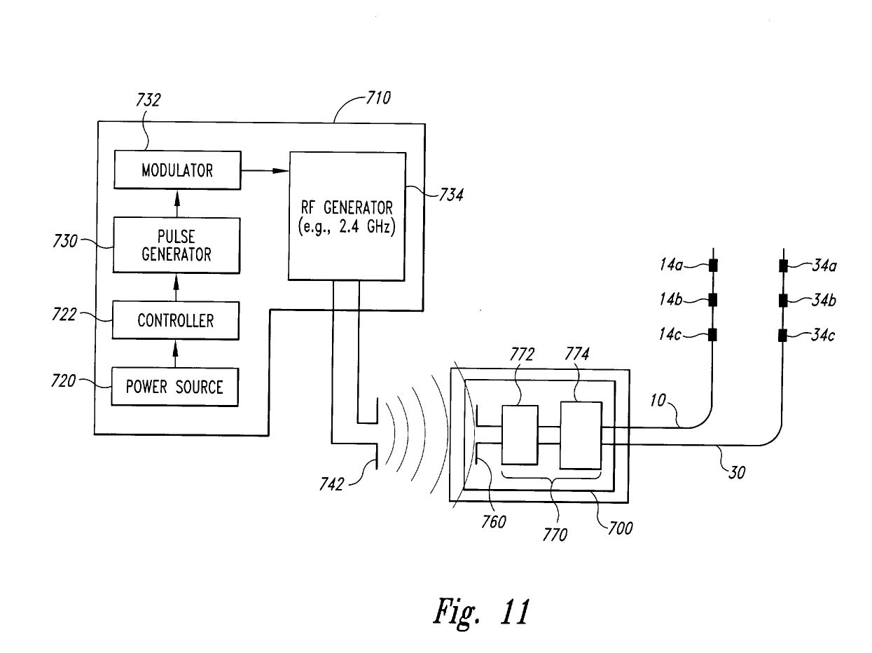

The neurostimulator 1 of FIG. 1 employs an extracorporeal pulse system 60. As many applications of the prior embodiment to treat wounds W will be relatively short-lived, typically extending a few weeks or less, utilizing external pulse system may be acceptable. The neurostimulator 2 of FIG. 2 employs a subcutaneously implantable pulse system 70, which may be implanted in the patient's abdomen, for example. This enables long-term treatment without having any connections that extend through the patient's skin S. As discussed below in connection with FIGS. 10 and 11, the implantable pulse system 70 may include its own power source or may be coupled to a simple external power source or controller. This can reduce the number of trips to the physician's office or clinic for treatment sessions, increasing patient independence.

The electrodes 10 and 30 in this embodiment may be subcutaneously implanted in much the same manner discussed above in connection with FIG. 1. Rather than being positioned on opposite sides of the wound W, though, the electrodes 10 and 30 may be positioned to extend along different sides of the patient's spinal column C outside the epidural space. In one embodiment of the invention, each of the contacts 14 and 34 are spaced about 2.5-10 cm from the midline of the spinal column C. As consequence, each of the contacts 14 of the first electrode 10 may be spaced at least 5 cm away from each of the contacts 34 of the second electrode 30. In one adaptation of this embodiment, each of the contacts 14 of the first electrode 10 are spaced about 5-20 cm from the nearest contact 34 of the second electrode 30. A range of 6-12 cm between the contacts 14 of the first electrode and the nearest contact 34 of the second electrode 30 should suffice for many applications.

Once the distal lengths of the electrodes 10 and 30 are implanted to position the contacts 14 and 34 at the desired locations, the remaining lengths of the electrodes 10 and 30 may be tunneled beneath the patient's skin S and electrically coupled to the pulse system 70 implanted in the patient's abdomen.

Once the neurostimulator 2 is implanted, the pulse system 70 can be used to deliver electrically therapy through the electrodes 10 and 30 as desired. The pulse system 70 could operate continuously without any external intervention. In the embodiments discussed below in connection with FIGS. 10 and 11, however, the pulse system 70 communicates with an external controller, which enables treatment sessions of varying lengths to be initiated as needed.

Before implanting the electrodes 10 and 30, the physician may identify a target tissue volume T, which includes one or more nerves targeted for neurostimulation. For example, the targeted nerve may comprise a nerve root at the dermatomal level corresponding to the patient's pain symptoms. In some embodiments of the invention, more than one target nerve may be selected. In the embodiment of FIG. 2, the contacts 14 and 34 are positioned to treat several dermatomal levels to treat pain in a broader area.

The precise location of any given nerve within a patient's body will vary from one patient to the next. For example, dorsal roots may follow different paths in different patients after exiting the spinal column C. In conventional PNS techniques, a physician would have to invasively cut away the patient's surrounding tissue to find the desired target nerve and place an electrode in direct physical contact with that nerve. In accordance with embodiments of the present invention, however, the physician need not identify the precise location of the target nerve in a particular patient. Instead, the physician can simply subcutaneously implant the electrode at an indeterminate distance from the target nerve within a tissue volume T encompassing the target nerve. By delivering electrical pulses to the contacts 14 and 34 to establish an electrical field within the targeted tissue volume T, the targeted nerve will be electrically stimulated.

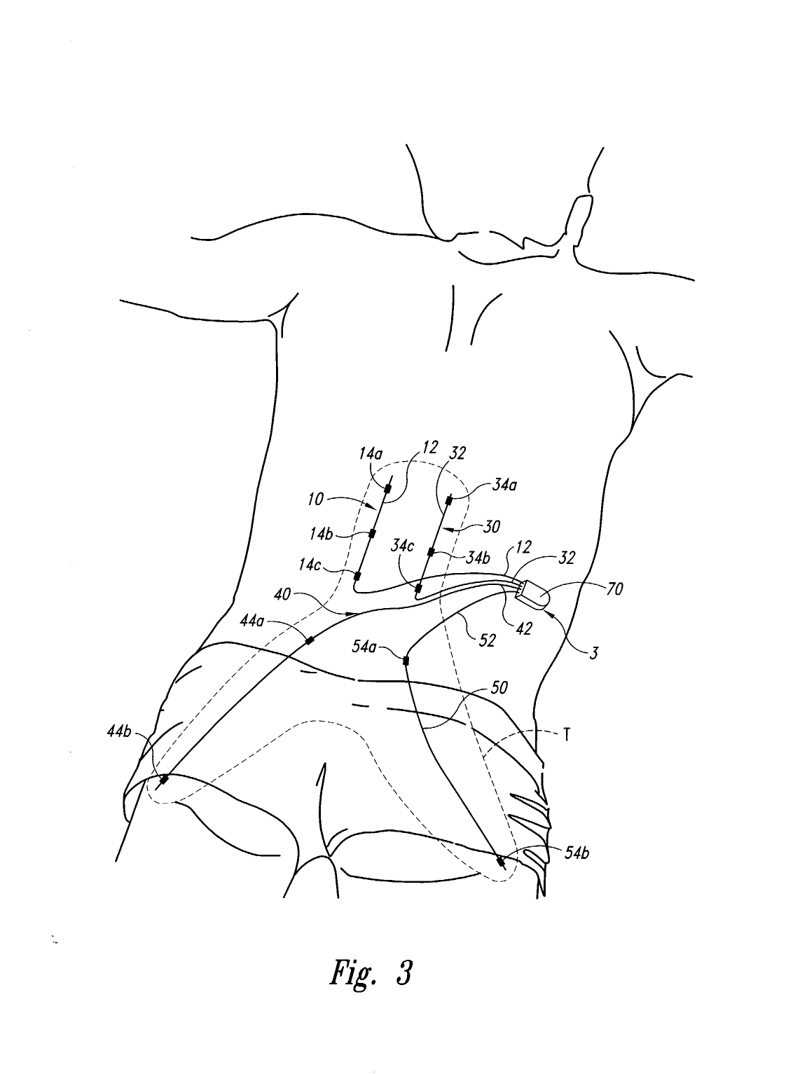

FIG. 3 schematically illustrates an application of a modified embodiment of the invention. In this embodiment, neurostimulator 3 may employ electrodes 10 and 30 and pulse system 70 which are implanted generally as outlined above in connection with FIG. 2. In FIG. 3, however, the pulse system 70 is connected to two additional electrodes. In particular, a third electrode 40 has a body 42 with a pair of contacts 44a-b spaced along its length. Similarly, the fourth electrode 50 has a body 52 with contacts 54a-b spaced along its length. The contacts 44 and 54 of the third and fourth electrodes 40 and 50 can be positioned to electrically stimulate additional target nerves, extending the target tissue volume T to cover an even broader area.

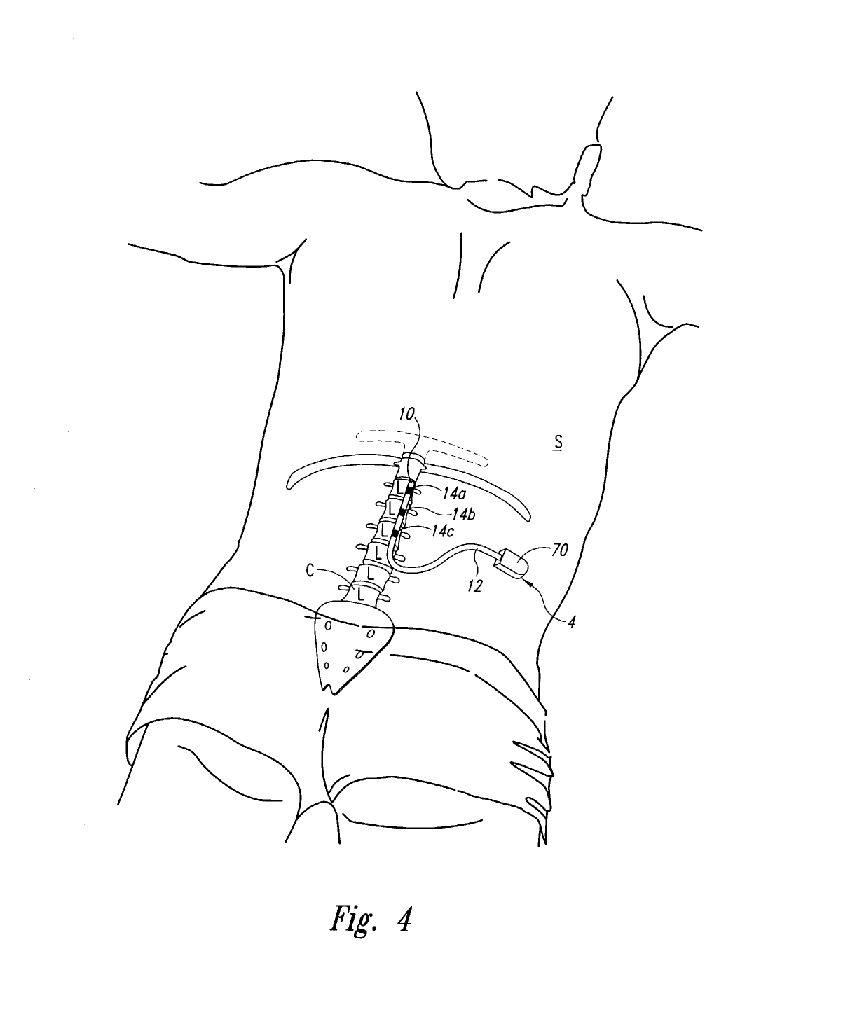

Each of the preceding embodiments employs two or more electrodes coupled to a common pulse system. In an alternative embodiment of the invention, however, a single electrode with multiple contacts may be employed. One specific application of this embodiment is shown schematically in FIG. 4, wherein the neurostimulator 4 includes a single electrode 10 having a body 12 and three electrical contacts 14a-c spaced along the length of the body 12. The electrode is coupled to a pulse system 70, which may be implanted beneath the skin S. In one embodiment, the pulse system 70 is adapted to deliver electrical pulses to the contacts 14a-c to generate an electrical field between the contacts, e.g., by generating an electrical potential between the contacts.

The single electrode 10 may be positioned along a midline of the spinal column C outside the epidural space, In the illustrated embodiment, the electrode extends dorsally of the spinal column generally alongside the spinous process. In other embodiments, the electrode 10 may be laterally spaced from the spinal column in a tissue volume containing a specified target nerve, e.g., a dorsal root exiting a specific level of the spinal column.

B. Selected Embodiments of Subcutaneously Implantable Electrodes for Use in Electrical Therapy

The neurostimulators 1-4 of FIGS. 1-4, respectively, can employ any of a wide variety of electrode styles. FIGS. 5-9 illustrate electrodes in accordance with different embodiments of the invention, which may be used in connection with the neurostimulators 1-4 discussed above.

FIG. 5 illustrates an electrode 100 in accordance with one embodiment of the invention. The electrode 100 includes a solid core wire 110, which has a dielectric cladding 120 covering much of the length of the core wire 110. The core wire 110 may be formed of any conductive bio-compatible material. A bio-compatible metal, e.g., surgical grade stainless steel, should work well for many applications. The dielectric cladding can be formed of a bio-compatible plastic or the like which will serve to electrically insulate unexposed lengths of the core wire 110 from surrounding tissue. The core wire 110 may lend the electrode 100 sufficient pushability to allow it to be advanced through the tissue volume T for deployment. To facilitate this advancement through the patient's tissue, the electrode 100 may be provided with a sharpened distal tip 140.

The dielectric cladding has gaps along its length to define two spaced-apart contacts 130a and 130b. Each of the contacts 130a-b may comprise an exposed length of the core wire 110 wherein the dielectric cladding 120 has been stripped away. Since the contacts 120 are exposed lengths of a common conductive core wire 110, both contacts 120 will have essentially the same electrical potential in use. The contacts 130a-b may be spaced from one another a distance l along the length of the electrode 100. The distance l between the contacts 130a-b may be the same as the lengths l₁-l₃ discussed above in connection with FIG. 1, e.g., 4-10 cm. While FIG. 5 illustrates two contacts 130a-b, the electrode 100 may include more or fewer electrodes for different applications.

FIGS. 6A-B illustrate an electrode 200 in accordance with an alternative embodiment of the invention. This electrode 200 includes a core wire 210 bearing a dielectric cladding 220. The core wire 210 and cladding 220 may be similar to the core wire 110 and cladding 120 of FIG. 5. The electrode 200 includes a first radial contact plate 230a and a second radial contact plate 230b spaced a distance l from one another. As a best seen in FIG. 6B (which shows only one of the contact plates 230a), the contact plate 230 may include a plurality of inwardly extending teeth 232. These teeth may pierce the dielectric cladding 220 and be embedded slightly in the core wire 210. This will electrically couple the contact plates 230a-b to the core wire 210 to enable delivery of an electrical impulse to tissue via the contact plates 230a-b. It is anticipated that the electrode 200 may be delivered through a trocar having a lumen diameter at least as great as the outer diameter of the contact plates 230a-b. When the trocar is withdrawn, the tissue will surround the contact plates 230a-b, providing good electrical contact between the contact plates 230a-b and the surrounding tissue and helping to anchor the electrode 200 in the tissue.

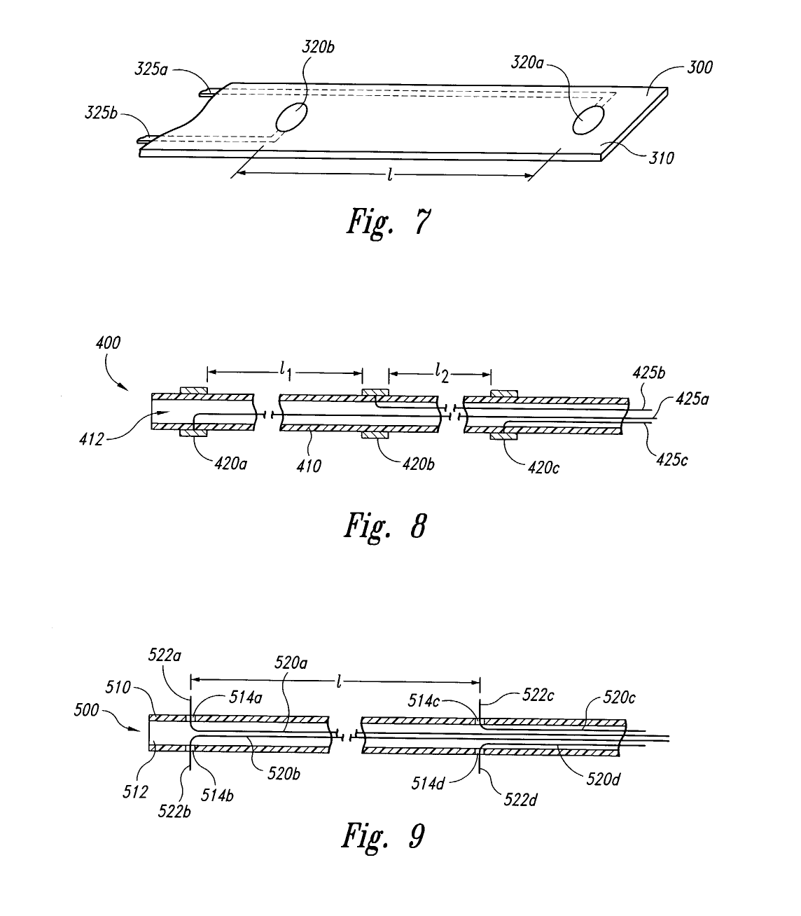

FIG. 7 illustrates an electrode 300 in accordance with yet another embodiment of the invention. This electrode 300 includes a flexible dielectric substrate 310 carrying a pair of contacts 320a-b spaced a distance l from one another. In this embodiment, the first contact 320a is electrically coupled to a first conductor 325a and the second contact 320b is coupled to a second conductor 325b. The conductors 325a and 325b may extend back the proximal end (not shown) of the electrode 300 for separate electrical connection to the pulse system. The conductors 325 are desirably covered with a dielectric layer or embedded in the flexible substrate 310 between the contacts 320 and the point at which they are connected to the pulse system. The electrode 300 may be manufactured in a manner similar to flexible printed circuit assemblies that are used in electronic devices. Such an electrode 300 may be rolled laterally into a long, thin structure which can be received in a lumen of a trocar (not shown) for implantation.

FIG. 8 illustrates yet another electrode 400 in accordance with a different embodiment of the invention. The electrode 400 includes a flexible dielectric body 410 and three contacts 420a-c. The body 410 may be formed of a suitable flexible biocompatible material, such as silicone or polyurethane. The body 410 may be solid, but the illustrated embodiment employs a body 410 having a central lumen 412. Each of the contacts 420a-c may comprise a conductive ring carried on an external surface of the body 410. The first and second contacts 420a-b may be spaced from one another a length l₁ along the body 410 while the second and third contacts 420b-c may be spaced a length l₂ along the body 410. Each of the contacts 420a-c may be electrically coupled to a separate conductor 420c. These conductors 420a-c may extend proximally along a length of the body 410 for connection to a pulse system (not shown). The pulse system may deliver different electrical pulses (e.g., pulses of different polarity) to the contacts 420a-c, creating an electrical potential between the contacts 420a-c. FIG. 8 illustrates the conductors 425a-c carried within the lumen 412 of the body 410. If so desired, these conductors 425a-c may instead by embedded in the wall of the body 410, facilitating advancement of a stylet through the lumen 412 for implanting the electrode 400, as discussed above.

FIG. 9 illustrates an electrode 500 in accordance with still another embodiment of the invention. The electrode 500 includes a flexible dielectric body 510 having a lumen 512. The body 510 of the electrode 500 may be similar to the body 410 of the electrode 400 in FIG. 8, but the body 510 includes a first pair of radially extending ports 514a-b extending through the wall at a distal location and a second pair of radially extending ports 514c-d spaced a length l along the body 510 proximally from the distal ports 514a-b. Each of the ports 514a-d is adapted to permit a distal tip 522a-d of a conductive wire 520a-d to pass therethrough. In one embodiment, these wires 520a-d are selectively deployable by manipulation at their proximal end. During implantation, the wires 520a-d may be retracted proximately so that the distal tips 522 extend only slightly, if at all, through the wall of the body 510. Once the body 510 is in position within the tissue volume T, the wires 520a-d may be advanced distally, urging the distal tips outwardly 522a-d through their respective radial ports 514a-d. This will embed the distal ends 522a-d of the wires 520a-d in the tissue volume, ensuring good electrical contact between the conductive distal ends 522 and the surrounding tissue and helping anchor the electrode 500 in place. If it is desired to remove the electrode 500 (e.g., at the end of the wound therapy discussed above in connection with FIG. 1), the wires 520a-d may be withdrawn proximally, retracting the distal tips 522a-d to facilitate removal. Hence, in this embodiment, the wires 520a-d comprise both electrical context and temporary anchors for the electrode 500.

C. Selected Embodiments of Subcutaneously Implantable Pulse Systems for Electrical Therapy

FIG. 10 schematically illustrates an integrated pulse system 70 in accordance with one embodiment of the invention which may be subcutaneously implanted as discussed above, e.g., in connection with the neurostimulator 2 of FIG. 2. The pulse system 70 can include a power supply 610, an integrated controller 620, a pulse generator 630, and a pulse transmitter 640. The power supply 610 can be a primary battery, such as a rechargeable battery or another suitable device for storing electrical energy. In alternative embodiments, the power supply 610 can be an RF transducer or a magnetic transducer that receives broadcast energy emitted from an external power source and converts the broadcast energy into power for the electrical components of the pulse system 70.

The integrated controller 620 can be a wireless device that responds to command signals sent by an external controller 650. The integrated controller 620, for example, can communicate with the external controller 650 by RF or magnetic links. The integrated controller 620 provides control signals to the pulse generator 630 in response to the command signals sent by the external controller 650. The pulse generator 630 can have a plurality of channels that send appropriate electrical pulses to the pulse transmitter 640, which is coupled to the electrodes 10 and 30. (The stylized electrodes 10 and 30 of FIG. 2 are shown here, but it should be understood that any of the electrodes 100-500 of FIGS. 5-9 could be employed instead of the schematically illustrated electrodes 10 and 30.) If each of the contacts 14 and 34 is provided with a separate conductor (e.g., conductors 425a-c in FIG. 8), the pulse generator 630 may have a separate channel for each of the conductors. Suitable components for the power supply 610, the integrated controller 620, the pulse generator 630, and the pulse transmitter 640 are known to persons skilled in the art of implantable medical devices.

FIG. 11 is a schematic view illustrating an embodiment of a pulse system 700, which may be used in place of the pulse system 70 of FIGS. 2 and 3, and an external controller 710 for controlling the pulse system 700 remotely from the patient using RF energy. In this embodiment, the external controller 710 includes a power supply 720, a controller 722 coupled to the power supply 720, and a pulse generator 730 coupled to the controller 722. The external controller 710 can also include a modulator 732. In operation, the external controller 710 broadcasts pulses of RF energy via an antenna 742.

In one embodiment, the pulse system 700 includes an antenna 760 and a pulse delivery system 770. The antenna 760 incorporates a diode (not shown) that rectifies the broadcast RF energy from the antenna 742. The pulse delivery system 770 can include a filter 772 and a pulse former 774 that forms electrical pulses that correspond to be RF energy broadcast from the antenna 742. The pulse system 700 is accordingly powered by the RF energy in the pulse signal from the external controller 710 such that the pulse system 700 does not need a separate power supply.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

1. An implantable neurostimulator adapted to treat pain, comprising:
a body-implantable first electrode including an elongate, flexible first conductor, a flexible, biocompatible first dielectric, and two exposed electrical contacts spaced from one another by at least about 3 cm along a length of the first electrode, the contacts being configured to deliver electrical stimulation to target tissue in which the first electrode is implanted;
a body-implantable second electrode including an elongate, flexible second conductor, a flexible, biocompatible second dielectric, and two exposed electrical contacts spaced from one another by at least about 3 cm along a length of the second electrode, the contacts being configured to deliver electrical stimulation to target tissue in which the second electrode is implanted, the second electrode being implantable at a location spaced at least about 5 cm from the first electrode; and
a common pulse system associated with the first and second electrodes, the pulse system being configured to deliver a current of at least about 0.6 mA to the first and second electrodes in a waveform efficacious to reduce a sensation of pain in nerves positioned at a location in the target tissue spaced from the first lead and from the second lead.

2. The neurostimulator of claim 1 wherein the electrical contacts of the first electrode are spaced from one another by no more than 15 cm.

3. The neurostimulator of claim 2 wherein the electrical contacts of the second electrode are spaced from one another by no more than 15 cm.

4. The neurostimulator of claim 2 wherein the electrical contacts of the first electrode are spaced from one another by 4-10 cm.

5. The neurostimulator of claim 4 wherein the electrical contacts of the second electrode are spaced from one another by 4-10 cm.

6. The neurostimulator of claim 2 wherein the electrical contacts of the first electrode are spaced from one another by 4-5 cm.

7. The neurostimulator of claim 6 wherein the electrical contacts of the second electrode are spaced from one another by 4-5 cm.

8. The neurostimulator of claim 1 wherein the electrical contacts of the first electrode comprise exposed lengths of the first conductor, the first electrode electrical contacts having the same electrical potential in use.

9. The neurostimulator of claim 8 wherein the electrical contacts of the second electrode comprise exposed lengths of the second conductor, the second electrode electrical contacts having the same electrical potential in use.

10. The neurostimulator of claim 1 wherein each contact of the first electrode is associated with a separate conductor.

11. The neurostimulator of claim 10 wherein each contact of the second electrode is associated with a separate conductor.

12. The neurostimulator of claim 1 wherein the first dielectric comprises a tubular member having a lumen within which the first conductor is received.

13. The neurostimulator of claim 1 wherein the pulse system comprises an implantable pulse generator, the first and second electrodes being coupled to the pulse generator.

14. The neurostimulator of claim 1 wherein the pulse system comprises an implantable receiver and an implantable pulse former, further comprising an extracorporeal pulse generator configured to broadcast energy to the receiver.

15. An implantable neurostimulator, comprising:
a body-implantable first electrode comprising an elongate first body carrying a first electrical contact and a second electrical contact, the first and second contacts being spaced from one another by 3-15 cm along a length of the first body, each of the first and second contacts being exposed for electrical contact with target tissue in which the first electrode is implanted;
a body-implantable second electrode comprising an elongate second body carrying a third electrical contact and a fourth electrical contact, the third and fourth contacts being spaced from one another by 3-15 cm along a length of the second body, each of the third and fourth contacts being exposed for electrical contact with the target tissue in which the second electrode is implanted; and
an implantable pulse system being configured to deliver electrical pulses to the first electrode and to the second electrode in a controlled waveform selected to stimulate nerves positioned at a location in the target tissue spaced from the first lead and from the second lead.

16. The neurostimulator of claim 15 wherein the electrical contacts of the first electrode are spaced from one another by 4-10 cm.

17. The neurostimulator of claim 16 wherein the electrical contacts of the second electrode are spaced from one another by 4-10 cm.

18. The neurostimulator of claim 15 wherein the electrical contacts of the first electrode are spaced from one another by 4-5 cm.

19. The neurostimulator of claim 18 wherein the electrical contacts of the second electrode are spaced from one another by 4-5 cm.

20. The neurostimulator of claim 15 wherein the first and second electrical contacts are coupled to a common first conductor of the first electrode and have the same electrical potential in use.

21. The neurostimulator of claim 20 wherein the third and fourth electrical contacts are coupled to a common second conductor of the second electrode and have the same electrical potential in use.

22. The neurostimulator of claim 15 wherein the first and second electrical contacts are coupled to separately controllable channels of the pulse source in use.

23. The neurostimulator of claim 22 wherein the third and fourth electrical contacts are coupled to a separately controllable channels of the pulse source in use.

24. The neurostimulator of claim 15 wherein the pulse system comprises an implantable pulse generator, the first and second electrodes being coupled to the pulse generator.

25. The neurostimulator of claim 15 wherein the pulse system comprises an implantable receiver and an implantable pulse former, further comprising an extracorporeal pulse generator configured to broadcast energy to the receiver.

26. A method of delivering electrical therapy to a recipient, comprising:
identifying a target volume of the recipient's body, the target volume including at least one target nerve positioned outside the patient's epidural space;
subcutaneously implanting a flexible first electrode having spaced-apart first and second contacts, the first and second contacts being positioned within the target volume at an indeterminate distance from the target nerve; and
electrically stimulating the target nerve by delivering a series of electrical pulses to the first electrode.

27. The method of claim 26 further comprising subcutaneously implanting a flexible second electrode having spaced-apart third and fourth contacts, the third and fourth contacts being positioned within the target volume at an indeterminate distance from the target nerve.

28. The method of claim 27 wherein the second electrode is implanted with the third contact spaced at least about 5 cm from each of the first and second contacts.

29. The method of claim 27 further comprising coupling the first and second electrodes to a common pulse system and delivering a series of electrical pulses to the second electrode.

30. The method of claim 29 wherein the common pulse system delivering the series of electrical pulses to the first electrode and the series of electrical pulses to the second electrode.

31. The method of claim 26 wherein delivering a series of electrical pulses to the first electrode comprises generating an electrical field between the first and second contacts.

32. The method of claim 26 further comprising coupling the first electrode to a pulse system.

33. The method of claim 32 wherein the pulse system comprises a power source and a pulse generator, further comprising implanting the pulse system in the recipient's body.

34. The method of claim 32 wherein the pulse system comprises a receiver and a pulse former, further comprising implanting the pulse system in the recipient's body.

35. The method of claim 34 wherein delivering a series of electrical pulses comprises receiving with the receiver a pulse of broadcast energy generated by an extracorporeal pulse generator and converting the broadcast energy into an electrical pulse with the pulse former.

36. The method of claim 26 wherein the electrical pulses are controlled to reduce a sensation of pain in the target nerve.

37. The method of claim 26 wherein the first electrode is implanted with the first contact positioned in subcutaneous fat.

38. The method of claim 26 wherein the first electrode is implanted with the first contact positioned in muscle tissue.

39. A method of delivering electrical therapy to a recipient, comprising:
identifying a target volume of the recipient's body, the target volume including at least one target nerve positioned outside the patient's epidural space;
subcutaneously implanting a flexible first electrode with a first contact within the target volume at an indeterminate distance from the target nerve;
subcutaneously implanting a flexible second electrode with a second contact within the target volume at an indeterminate distance from the target nerve, but with the second contact spaced at least about 5 cm from the first contact;
coupling the first electrode and the second electrode to a common pulse system; and
electrically stimulating the target nerve by generating an electrical field between the first and second electrodes.

40. The method of claim 39 wherein the pulse system comprises a power source and a pulse generator, further comprising implanting the pulse system in the recipient's body.

41. The method of claim 40 wherein generating an electrical field between the first and second electrodes comprises delivering electrical pulses to the first electrode and to the second electrode from the pulse generator.

42. The method of claim 39 wherein the pulse system comprises a receiver and a pulse former, further comprising implanting the pulse system in the recipient's body.

43. The method of claim 42 wherein generating an electrical field between the first and second electrodes comprises receiving with the receiver a pulse of broadcast energy generated by an extracorporeal pulse generator and converting the broadcast energy into an electrical pulse with the pulse former.

44. The method of claim 39 wherein the electrical field is controlled to reduce a sensation of pain in the target nerve.

45. The method of claim 39 wherein the first electrode is implanted with the first contact positioned in subcutaneous fat.

46. The method of claim 45 wherein the second electrode is implanted with the second contact positioned in subcutaneous fat.

47. The method of claim 39 wherein the first electrode is implanted with the first contact positioned in muscle tissue.

48. The method of claim 47 wherein the second electrode is implanted with the second contact positioned in muscle tissue.

49. A method of electrically stimulating a plurality of nerves distributed in a target volume of a recipient's body, comprising:
subcutaneously implanting a first electrode at a first location, the first electrode defining at least two first electrical contact sites within the target volume spaced along the length of the first electrode;
subcutaneously implanting a second electrode at a second location, the second electrode defining at least two second electrical contact sites within the target volume spaced along the length of the second electrode, the contact sites of the second electrode being spaced at least about 5 cm from the contact sites of the first electrode;
coupling the first electrode and the second electrode to a common pulse system;
subcutaneously implanting the pulse system in the recipient's body; and
electrically stimulating the plurality of nerves by delivering electrical pulses to the first and second electrodes via the pulse system.

50. The method of claim 49 wherein the pulse system comprises a power source and a pulse generator, the pulse generator delivering the electrical pulses to the first and second electrodes.

51. The method of claim 49 wherein the pulse system comprises a receiver and a pulse former, delivering electrical pulses to the first and second electrodes comprising receiving with the receiver a pulse of broadcast energy generated by an extracorporeal pulse generator and converting the broadcast energy into an electrical pulse with the pulse former.

52. The method of claim 49 wherein the electrical pulses are controlled to reduce a sensation of pain in at least one of the plurality of nerves.

53. The method of claim 49 wherein the first electrical contact sites are positioned in subcutaneous fat.

54. The method of claim 53 wherein the second electrical contact sites are positioned in subcutaneous fat.

55. The method of claim 49 wherein the first electrical contact sites are positioned in muscle tissue.

56. The method of claim 55 wherein the second electrical contact sites are positioned in muscle tissue.

57. A method of delivering electrical therapy to a recipient, comprising:
subcutaneously implanting an elongate first electrode at a location extending on a first side of the recipient's spinal column at a location outside the epidural space, the first electrode including a first contact and a second contact, the second contact being spaced at least 3 cm from the first contact; and
electrically stimulating nerves in the patient's back by delivering electrical pulses to the first and second contacts.

58. The method of claim 57 further comprising coupling the first electrode to a common pulse system and delivering the electrical pulses to the first and second contacts via the pulse system.

59. The method of claim 58 further comprising implanting the pulse system in the recipient's body.

60. The method of claim 58 wherein the pulse system comprises a power source and a pulse generator, further comprising implanting the pulse system in the recipient's body.

61. The method of claim 58 wherein the pulse system comprises a power delivery system having a receiver and a pulse former, further comprising implanting the pulse system in the recipient's body.

62. The method of claim 61 wherein delivering electrical pulses to the first and second contacts comprises receiving with the receiver pulses of broadcast energy generated by an extracorporeal pulse generator and converting the broadcast energy into electrical pulses with the pulse former.

63. The method of claim 57 wherein the electrical pulses are controlled to reduce a sensation of pain in at least one of the nerves.

64. A method of delivering electrical therapy to a recipient, comprising:
subcutaneously implanting an elongate first electrode at a location extending on a first side of the recipient's spinal column at a location outside the epidural space, the first electrode including a first contact and a second contact, the second contact being spaced at least 3 cm from the first contact;
subcutaneously implanting an elongate second electrode at a location extending on a second side of the recipient's spinal column at a location outside the epidural space, the second electrode including a third contact and a fourth contact, the fourth contact being spaced at least 3 cm from the third contact, the second side being different from the first side and the third and fourth contacts being spaced from each of the first and second contacts by at least about 5 cm; and
electrically stimulating nerves in the patient's back by delivering electrical pulses to the first and second electrodes.

65. The method of claim 64 further comprising coupling the first electrode and the second electrode to a common pulse system and delivering the electrical pulses to the first and second electrodes via the pulse system.

66. The method of claim 65 further comprising implanting the pulse system in the recipient's body.

67. The method of claim 65 wherein the pulse system comprises a power source and a pulse generator, further comprising implanting the pulse system in the recipient's body.

68. The method of claim 65 wherein the pulse system comprises a power delivery system having a receiver and a pulse former, further comprising implanting the pulse system in the recipient's body.

69. The method of claim 68 wherein delivering electrical pulses to the first and second electrodes comprises receiving with the receiver pulses of broadcast energy generated by an extracorporeal pulse generator and converting the broadcast energy into electrical pulses with the pulse former.

70. The method of claim 64 wherein the electrical pulses are controlled to reduce a sensation of pain in at least one of the nerves.

71. The method of claim 64 wherein the first electrode is implanted with the first and second contacts positioned in subcutaneous fat.

72. The method of claim 71 wherein the second electrode is implanted with the third and fourth contacts positioned in subcutaneous fat.

73. The method of claim 64 wherein the first electrode is implanted with the first and second contacts positioned in muscle tissue.

74. The method of claim 73 wherein the second electrode is implanted with the third and fourth contacts positioned in muscle tissue.

75. A method of electrically treating a wound, comprising:
subcutaneously implanting a first electrode along a first side of the wound;
subcutaneously implanting a second electrode along a second side of the wound opposite the first side of the wound;
coupling the first and second electrodes to a common pulse system; and
electrically stimulating nerves in a tissue volume including the wound by delivering electrical pulses to the first and second electrodes.

76. The method of claim 75 wherein the wound comprises a surgical incision made in the course of a surgical procedure, the nerves being stimulated during a postoperative period following the surgical procedure.

77. The method of claim 75 wherein the first electrode comprises an elongate flexible body and spaced-apart first and second electrical contacts, the first electrode being implanted with the first and second electrical contacts spaced along the first side of the wound.

78. The method of claim 77 wherein the second electrode comprises an elongate flexible body and spaced-apart third and fourth electrical contacts, the second electrode being implanted with the third and fourth electrical contacts spaced along the second side of the wound.

79. The method of claim 75 further comprising coupling the first electrode and the second electrode to a common pulse system and delivering the electrical pulses to the first and second electrodes via the pulse system.

80. The method of claim 79 further comprising implanting the pulse system in the recipient's body.

81. The method of claim 79 wherein the pulse system comprises a power source and a pulse generator, further comprising implanting the pulse system in the recipient's body.

82. The method of claim 79 wherein the pulse system comprises a power delivery system having a receiver and a pulse former, further comprising implanting the pulse system in the recipient's body.

83. The method of claim 82 wherein delivering electrical pulses to the first and second electrodes comprises receiving with the receiver pulses of broadcast energy generated by an extracorporeal pulse generator and converting the broadcast energy into electrical pulses with the pulse former.

84. The method of claim 75 wherein the electrical pulses are controlled to reduce a sensation of pain in at least one of the nerves.

85. The method of claim 75 wherein the first electrode is implanted with a first electrical contact positioned in subcutaneous fat.

86. The method of claim 85 wherein the second electrode is implanted with a second electrical contact positioned in subcutaneous fat.

87. The method of claim 75 wherein the first electrode is implanted with a first electrical contact positioned in muscle tissue.

88. The method of claim 87 wherein the second electrode is implanted with a second electrical contact positioned in muscle tissue.
